(19) United States
(12) Patent Application Publication
(10) Pub. No.: US 2005/0191619 A1
(43) Pub. Date: Sep. 1, 2005

(54) DNA purification and recovery from high particulate and solids samples

(75) Inventors: James C. Davis, Kingston, MA (US); Martin A. Smith, Newton, MA (US); Frank D. Igoe, Walpole, MA (US); Marcela A. Vera-Garcia, Framingham, MA (US)

Correspondence Address: EDWARDS & ANGELL, LLP, P.O. BOX 55874, BOSTON, MA, 02205, US

(73) Assignee: Whatman, Inc.

(21) Appl. No.: 10/997,647

(22) Filed: Nov. 23, 2004

Related U.S. Application Data
(62) Division of application No. 10/224,129, filed on Aug. 20, 2002.
(60) Provisional application No. 60/313,767, filed on Aug. 20, 2001.

(51) Int. Cl. C12Q 1/70; C12Q 1/68; C07H 21/04; A61M 35/00

(52) U.S. Cl. 435/5; 435/6; 435/270; 536/25.4; 604/1

(57) ABSTRACT

This invention relates to methods for rapid nucleic acid purification from sources heavily contaminated with high particulate material, such as cellular debris, and solids, including suspended solids. In particular, this invention provides methods for rapid, quantifiable recovery and purification of nucleic acids from a variety of sources heavily contaminated with solids, such as small organisms, tissue samples, samples of blood found on soil, or samples of washing from foods, which are frequently difficult sources for nucleic acid isolation due to their propensity to clog filters and columns. A device and kit are also provided.

### CROSS-REFERENCE TO RELATED APPLICATIONS

This application is a divisional of U.S. Ser. No. 10/224,129, filed Aug. 20, 2002 which claims priority from U.S. Provisional Application Ser. No. 60/313,767, filed Aug. 20, 2001.

### FIELD OF THE INVENTION

This invention relates to methods, a device, and a kit for rapid nucleic acid purification from sources heavily contaminated with high particulate material, such as cellular debris, soil, and solids, including suspended solids, and from mixtures of cells.

### BACKGROUND OF THE INVENTION

It is well known that some sources of nucleic acid in a variety of matrices include cellular material, soil, and other solids that complicate nucleic acid purification and rapid isolation. These also include complex mixtures or suspensions containing more than one cell type. It is also known that the use of filters in centrifuge spin baskets can result in severe plugging of the filters with the particulates and cellular debris resulting in loss of sample and incomplete purification and recovery. Simple pre-filtration can often improve the process, but unless the target sample is concentrated afterwards, there is no significant advantage in terms of time, recovery, reagents, and reproducibility. Some nucleic acid isolation products, such as spin tubes, have some degree of usefulness, but are still subject to serious limitations, such as clogging of filters or columns, when faced with high-suspended solids in the sample.

Some samples containing nucleic acids are of a type or source so as to make nucleic acid isolation procedures more difficult. In some instances, the sample may comprise a complex matrix, such as blood or semen found on soil, sand, or cloth, or cells, oocysts, or bacteria from washings of foods, or the sample may be a mixture containing multiple cell types. In other instances, samples from tissues or small organisms, even if pre-homogenized, may still contain a large amount of debris, such as extracellular matrix (“ECM”) components, lipids, or complex biological deposits. The complexity of these sample matrices presents formidable difficulties to nucleic acid purification. These types of samples routinely hinder nucleic acid isolation experiments in medicine, forensics, and basic research.

It would be useful to have methods for rapid purification of nucleic acid from sources heavily contaminated with high particulate material and from mixtures of cells. It would be useful to have a device or a kit for practicing these methods.

### SUMMARY OF THE INVENTION

This invention relates to methods and a device and a kit for rapid nucleic acid purification from sources heavily contaminated with high particulate material, such as cellular debris, and solids, including suspended solids, and from mixtures of cells.

In one aspect, the invention provides a method of isolating nucleic acids from a sample containing cells or viruses, comprising:
a. providing a dry solid medium comprising a composition containing a lysis agent;
b. contacting the medium on one surface with the sample;
c. lysing the cells or viruses and allowing components of the sample, comprising the nucleic acids, to enter the medium;
d. washing the medium from the opposite surface with a wash buffer; and
e. eluting the nucleic acid from the medium.

In another aspect, the invention provides a method of isolating nucleic acids from a sample containing cells or viruses, comprising:
a. providing a dry solid medium;
b. lysing the cells or viruses with a lysis agent;
c. contacting the medium on one surface with the lysed sample to allow components of the sample, comprising the nucleic acids, to enter the medium;
d. washing the medium from the opposite surface with a wash buffer; and
e. eluting the nucleic acid from the medium.

In another aspect, the invention provides a method of isolating nucleic acids from a sample, comprising:
a. providing a dry solid medium comprising a composition consisting essentially of an anionic surfactant or an anionic detergent;
b. contacting the medium on one surface with the sample to allow components of the sample, comprising the nucleic acids, to enter the medium;
c. washing the medium from the opposite surface with a wash buffer; and
d. eluting the nucleic acid from the medium.

In yet another aspect, the invention provides a method of isolating nucleic acid from a sample containing cells or viruses containing nucleic acid, comprising:
a. providing a pre-filter comprising a dense medium capable of retaining contaminants larger than the cells or viruses containing nucleic acid;
b. providing a size-exclusion barrier capable of retaining the cells or viruses containing nucleic acid;
c. contacting the pre-filter with the sample;
d. drawing the sample through the pre-filter so that the nucleic acid-containing cells or viruses are drawn through the filter;
e. contacting the size-exclusion barrier with the sample containing the nucleic acid-containing cells or viruses;
f. trapping the nucleic acid-containing cells or viruses on the size-exclusion barrier while drawing liquid components through the size-exclusion barrier; and
g. removing the trapped nucleic acid-containing cells or viruses from the filter.

In addition, the method may further comprise:
h. providing a dry solid medium comprising a composition containing a lysis agent;
i. contacting the nucleic acid-containing cells or viruses with the medium;
j. lysing the nucleic acid-containing cells or viruses and allowing components of the sample, comprising the nucleic acids, to enter the medium;
k. washing the medium; and
l. eluting the nucleic acid from the medium.

In still another aspect, the invention provides a device for separation of components of high particulate or complex samples containing cells or viruses containing nucleic acids, comprising:
a. a pre-filter comprising a dense medium capable of retaining contaminants larger than the cells or viruses containing nucleic acid;
b. a size-exclusion barrier capable of retaining cells or viruses containing nucleic acid; and
c. a connection between the pre-filter and the size-exclusion barrier capable of directing the sample from the pre-filter to the size-exclusion barrier.

In another aspect, the invention provides a kit for isolating nucleic acids from a sample, comprising:
a. a pre-filter comprising a dense medium capable of retaining contaminants larger than the cells or viruses containing nucleic acid;
b. a size-exclusion barrier capable of retaining cells or viruses containing nucleic acid;
c. a connection between the pre-filter and the size-exclusion barrier capable of directing the sample from the pre-filter and the size-exclusion barrier; and
d. a dry solid medium capable of retaining nucleic acid.

### BRIEF DESCRIPTION OF THE DRAWINGS

FIG. 1A depicts a cross-section of one type of device for filtration and sample Concentration according to one embodiment of the present invention. Arrows indicate the direction of sample flow.

FIG. 1B depicts an exploded view of the device of FIG. 1A. Arrows indicate the direction of sample flow.

FIG. 2 is an agarose gel photograph showing the detection of bacterial DNA (from a large number of cells) collected on a FTA™ filter and subjected to a polymerase chain reaction (PCR) with primers for the enolase gene product.

FIG. 3 is an agarose gel photograph showing the detection of DNA collected from different numbers of cells on a FTA™ filter and subjected to PCR with primers for the enolase gene product.

FIG. 4 is an agarose gel photograph showing the detection of DNA collected from different numbers of cells on a FTA™ filter and subjected to a first round of PCR with primers for the enolase gene product.

FIG. 5 is an agarose gel photograph showing the detection of DNA after a re-amplification of the products depicted in FIG. 4.

### DETAILED DESCRIPTION OF THE INVENTION

This invention provides methods, a device, and a kit for utilizing filter technology for rapid purification and elution of nucleic acids. In particular, this invention provides methods for rapid, quantifiable recovery and purification of nucleic acids from a variety of sources heavily contaminated with solids, multiple cell types, or other matter.

The present invention has many advantages, including the following:
1. It enables nucleic acid recovery from complex, less-processed samples.
2. It is useful for samples recovered from complex matrices, such as small organisms, tissues, or blood found on soil.
3. It produces rapid, reliable, reproducible results from various sample matrices.
4. In one embodiment, it enables improved efficiency of nucleic acid collection from high particulate and/or large volume samples by including a simple pre-processing of sample consisting of pre-filtration and concentration onto a permeable barrier.

Several aspects of the invention have been described in the Summary of the Invention.

In one embodiment, the lysis agent comprises an anionic surfactant or an anionic detergent. In another embodiment, the lysis agent comprises an anionic surfactant or an anionic detergent and:
i. a weak base;
ii. a chelating agent; and
iii. optionally uric acid or a urate salt.

In one embodiment, the dry solid medium comprises glass fiber, cellulose, or non-woven polyester, more preferably in the form of a swab or a filter.

In one embodiment, the dry solid medium comprises a composition consisting of a lysis agent. In another embodiment, the dry solid medium comprises a composition consisting essentially of an anionic surfactant or an anionic detergent. In yet another embodiment, the dry solid medium comprises a composition consisting essentially of an anionic surfactant or an anionic detergent and the composition further comprises:
i. a weak base;
ii. a chelating agent; and
iii. optionally uric acid or a urate salt.

In one embodiment, the eluting step further comprises
i. heating an elution buffer to an elevated temperature in the range of 40° C. to 125° C.; and
ii. contacting the medium with the heated elution buffer.

In another embodiment, the eluting step further comprises:
i. contacting the medium with an elution buffer; and
ii. heating the medium and the elution buffer to an elevated temperature in the range of 40° C. to 125° C.

In preferred embodiments, the elevated temperature is in the range of 80° C. to 95° C. More preferably, the elution buffer is heated to an elevated temperature of 80° C. to 95° C., added to the medium, and the medium and elution buffer are heated to an elevated temperature of 80° C. to 95° C., still more preferably for 10 minutes.

The nucleic acids preferably comprise DNA or RNA.

In one embodiment, the sample comprises a biological tissue or organ, a cell, a virus, a homogenate of a biological tissue or organ, blood, bile, pus, lymph, spinal fluid, feces, saliva, sputum, mucus, urine, discharge, tears, sweat, culture medium, water, wash water, or a beverage.

In one embodiment, the invention provides a method of isolating nucleic acid from a sample containing cells or viruses containing nucleic acid, comprising:
a. providing a pre-filter comprising a dense medium capable of retaining contaminants larger than the cells or viruses containing nucleic acid;
b. providing a size-exclusion barrier capable of retaining the cells or viruses containing nucleic acid;
c. contacting the pre-filter with the sample;
d. drawing the sample through the pre-filter so that the nucleic acid-containing cells or viruses are drawn through the filter;
e. contacting the size-exclusion barrier with the sample containing the nucleic acid-containing cells or viruses;
f. trapping the nucleic acid-containing cells or viruses on the size-exclusion barrier while drawing liquid components through the size-exclusion barrier; and
g. removing the trapped nucleic acid-containing cells or viruses from the filter.

In a preferred embodiment, the above method further comprises:
h. providing a dry solid medium comprising a composition containing a lysis agent;
i. contacting the nucleic acid-containing cells or viruses with the medium;
j. lysing the nucleic acid-containing cells or viruses and allowing components of the sample, comprising the nucleic acids, to enter the medium;
k. washing the medium; and
l. eluting the nucleic acid from the medium.

Preferably, the pre-filter further comprises glass microfiber, cellulose acetate, polypropylene, melt-blown polypropylene, scintered glass, or polyethylene.

Preferably, the size-exclusion barrier comprises a polycarbonate track-etch membrane.

In one embodiment, the invention provides a device for separation of components of high particulate or complex samples containing cells or viruses containing nucleic acids, comprising:
a. a pre-filter comprising a dense medium capable of retaining contaminants larger than the cells or viruses containing nucleic acid;
b. a size-exclusion barrier capable of retaining cells or viruses containing nucleic acid; and
c. a connection between the pre-filter and the size-exclusion barrier capable of directing the sample from the pre-filter to the size-exclusion barrier.

Preferably, the pre-filter further comprises glass microfiber, cellulose acetate, polypropylene, melt-blown polypropylene, scintered glass, or polyethylene.

Preferably, the size-exclusion barrier comprises a polycarbonate track-etch membrane.

In another embodiment, the invention provides a kit for isolating nucleic acids from a sample, comprising:
a. a pre-filter comprising a dense medium capable of retaining contaminants larger than the cells or viruses containing nucleic acid;
b. a size-exclusion barrier capable of retaining cells or viruses containing nucleic acid;
c. a connection between the pre-filter and the size-exclusion barrier capable of directing the sample from the pre-filter and the size-exclusion barrier; and
d. a dry solid medium capable of retaining nucleic acid.

In a preferred embodiment, the kit further comprises:
e. a lysis buffer;
f. a wash buffer; and
g. an elution buffer.

In one embodiment, the dry solid medium comprises a composition comprising a lysis agent.

In another embodiment, the dry solid medium comprises a composition containing an anionic surfactant or an anionic detergent.

In another embodiment, the dry solid medium comprises a composition containing an anionic surfactant or an anionic detergent and the composition further comprises:
i. a weak base;
ii. a chelating agent; and
iii. optionally uric acid or a urate salt.

Preferably, the dry solid medium comprises glass fiber, cellulose, or non-woven polyester.

Preferably, the dry solid medium is in the form of a swab or a filter.

Preferably, the pre-filter further comprises glass microfiber, cellulose acetate, polypropylene, melt-blown polypropylene, scintered glass, or polyethylene.

Preferably, the size-exclusion barrier comprises a polycarbonate track-etch membrane.

According to one embodiment of the present invention; a centrifuge tube with a filter is provided. One example of a centrifuge tube with a filter is a GenPrep™ spin tube containing an FTA™ Elute filter as a dry solid medium. Other types of FTA™ filter technology may also be utilized. A nucleic acid-containing sample is also provided, such as a sample comprising cells or pathogens.

According to one embodiment of the present invention, the nucleic acid-containing sample is applied to the bottom of the filter, rather than on the top of the filter, while the tube is inverted. The filter is preferably a glass fiber filter, a cellulose filter, or a non-woven polyester filter. More preferably, the filter is a glass fiber, cellulose, or non-woven polyester filter with an FTA™ coating. The cells are lysed, or the pathogens are inactivated. The lysate containing nucleic acids enters the matrix of fibers in the filter, which stabilizes and binds the nucleic acids. Once the lysate has entered the matrix of the filter, the loaded spin basket unit is placed in the centrifuge spin tube such that the filter is returned to its upright orientation and the cellular debris is now below the filter. Isolation of the nucleic acids proceeds, but because the solids from the cellular or pathogenic debris are below the filter in the bottom of the tube, they are largely eliminated from the tube during the first washing step and do not clog the filter. Nucleic acids are retained in the filter fibers, purified, and eluted.

Preferably, the filter comprises a glass fiber, cellulose, or non-woven polyester filter with a coating comprising a weak base, a chelating agent, an anionic surfactant or anionic detergent, and optionally uric acid or a urate salt. One commercial example of such a filter is the FTA™ filter or the FTA™ Elute filter in the GenPrep™ column (Whatman, Inc.). Preferably, the coating lyses cells or viral pathogens upon contact, thereby releasing the nucleic acids and other cellular components in the cell lysate, which enters the filter.

Other relevant disclosure is found in U.S. Pat. No. 5,496,562, dated Mar. 5, 1996, in U.S. Pat. No. 5,807,527, dated Sep. 15, 1998, in U.S. Pat. No. 5,756,126, dated May 26, 1998, all of which are incorporated herein by reference, and in related patents and patent applications.

According to another embodiment of the present invention, the nucleic acid containing sample contained in a complex mixture of cells and particulates is pre-filtered through a dense matrix to remove larger particulates and then concentrated on a size-exclusion barrier, where it is sequestered for collection. The sample is collected either by washing the surface of the size exclusion barrier with small amounts of isotonic neutral buffer for application to a medium, such as an FTA™ matrix (Whatman, Inc.), or by swabbing the surface of the size exclusion barrier with a small piece of the medium, such as an FTA™ matrix.

One embodiment of this invention includes a pre-filtration step followed by a target sample concentration step before the nucleic acid purification step. According to one embodiment of the invention, a device is provided for nucleic acid purification from complex samples, including large volumes (≧200 ml) of such samples.

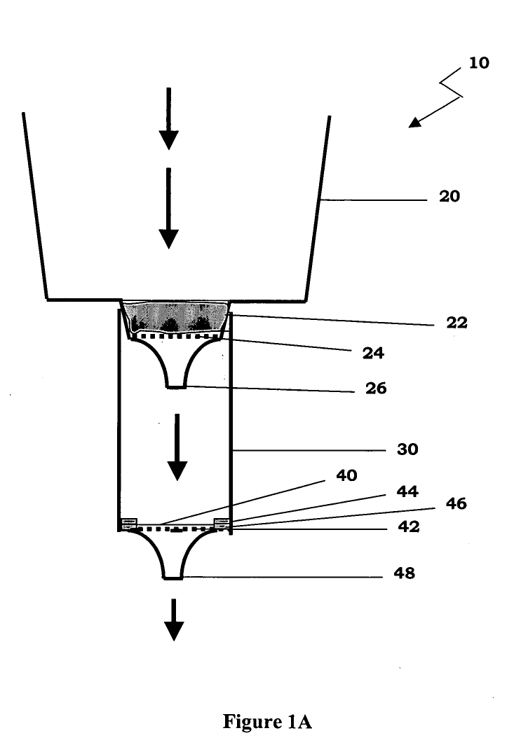

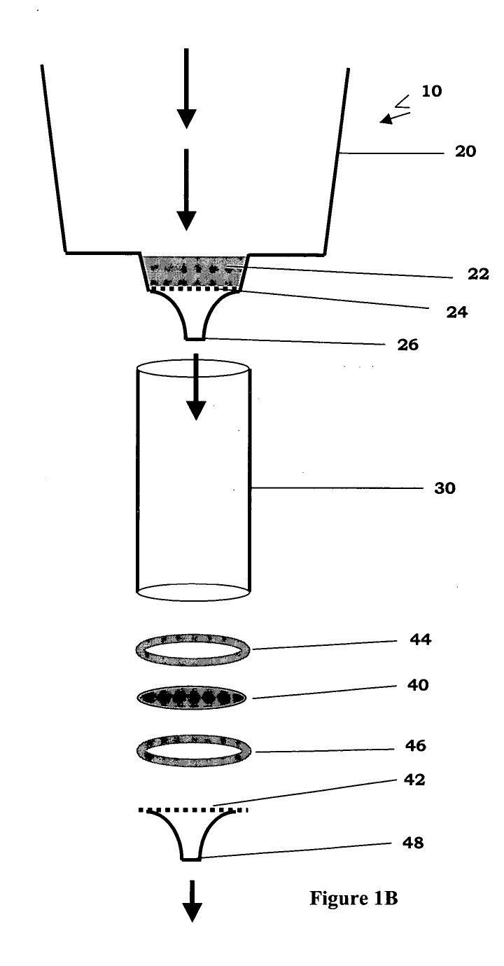

FIG. 1A depicts one type of device (10) for pre-filtration and sample concentration according to one embodiment of the invention. FIG. 1B depicts an exploded view of the device of FIG. 1A. It is understood by one of ordinary skill in the pertinent art that other types of devices are possible according to this embodiment of the invention. It is also understood that other types of devices are possible according to other embodiments of the invention.

In FIGS. 1A and 1B, the arrows indicate the direction of the sample flow. Preferably, a vacuum is applied to the device to improve the rate of flow. The upper funnel (20) contains the dense pre-filter (22), which is supported by a support (24). The sample is added to the upper funnel (20), and the large particulates, such as those found in soil, are trapped in the pre-filter (22), while the target sample flows through the pre-filter (22) and through the outlet (26) into the lower funnel (30) containing the small-pore membrane (size exclusion barrier) (40), which is supported by a support (42). Optionally, the small-pore membrane (size-exclusion barrier) is sandwiched between two ring-shaped or doughnut-shaped gaskets (44 and 46). The pre-filter (22) is washed with a small amount of isotonic buffer of neutral pH to minimize any retention of target sample. The small-pore membrane (40) acts as a size exclusion barrier, allowing the liquid to pass through the small-pore membrane (40) and the outlet (48), but trapping the particles, which include one or more of the following: cells, bacteria, viruses, oocysts, and other microbes, as well as other similar-sized particulates in suspension. The device may be disassembled and the sample collected from the surface of the small-pore membrane and applied to FTA™ as described in Example 6.

In one embodiment of the invention, the pre-filter comprises a dense medium capable of retaining contaminants larger than the cells or viruses of interest containing the nucleic acids. Preferred embodiments of the dense material include, but are not restricted to, glass microfiber filter, cellulose acetate filter, polypropylene filter, scintered glass or polyethylene filter. Preferred polypropylene filter is made from melt-blown polypropylene. It is most preferred that most of the cells or viruses of interest will be capable of passing through the dense medium, while the larger contaminants are retained by it.

In one embodiment of the invention, the size-exclusion barrier is capable of retaining the cells and viruses of interest containing the nucleic acids. Preferred embodiments of the size-exclusion barrier include, but are not limited to polycarbonate track-etch membranes. It is most preferred that most of the cells or viruses will be retained by the size exclusion barrier, while most of the smaller contaminants will be capable of passing through it.

In one embodiment, one or both of the outlets is a Luer outlet. Use of a Luer outlet (especially as shown in a position corresponding to the lower outlet (48) in FIG. 1) may aid in vacuum filtration if a vacuum is used.

Whole cells, cellular debris, viruses, and other biological material may be treated while being retained by the filter by the application of a detergent to the filter. Any detergent may be used, provided that it has the effect of rupturing or “peeling away” the cell membrane to leave nuclear material. The nucleic acid is retained by the filter. Preferably the detergent is selected from sodium dodecyl sulfate (particularly 0.5% weight-by-volume SDS), or other commercially available detergents such as TWEEN™ 20 (particularly 1% volume-by-volume TWEEN™ 20), LDS (particularly 1% w/v LDS) or TRITON™ e.g., TRITON™ X-100 (particularly 1% v/v TRITON™). The amount of detergent employed is sufficient to lyse cell membranes, but not so much as to denature DNA. Suitable amounts are generally 0.1% to 2% by weight (w/v) and preferably 0.2% to 1.5% w/v and more preferably 0.5% to 1.05% w/v.

While the addition of detergent is preferable, the present method may be carried out without the addition of a detergent by using other known lysing agents. However, applying a detergent to the cells or viruses while the cells or viruses are retained by the filter increases the yield and purity of the DNA product.

In addition to rupturing the intact whole cells to expose nucleic acids, the detergent also has the function of washing out protein, heme (haem), and other debris and contaminants which may have been retained by the filter.

Alternatively, the nucleic acids may be trapped on a dry solid medium, such as a filter, comprising a composition containing a lysis agent. Preferably, the “dry solid medium” as used herein means a porous material or filter media formed, either fully or partly from glass, silica or quartz, including their fibers or derivatives thereof, but is not limited to such materials. Other materials from which the filter membrane can be composed also include cellulose-based (nitrocellulose or carboxymethylcellulose papers), hydrophilic polymers including synthetic hydrophilic polymers (e.g. polyester, polyamide, carbohydrate polymers), polytetrafluoroethylene, and porous ceramics.

The media used for the filter membrane of the invention includes any material that does not inhibit the sorption of the chemical coating solution and which does not inhibit the storage and subsequent analysis of nucleic acid-containing material added to it. Preferably, the material does not inhibit elution of the nucleic acid and its subsequent analysis. This includes flat dry matrices or a matrix combined with a binder. It is preferred that the filter membrane of the invention be of a porous nature to facilitate immobilization of nucleic acid.

In embodiments wherein the dry solid medium comprises a composition containing a lysis agent, the composition of the lysis agent is preferably an anionic surfactant or an anionic detergent. Alternatively, the lysis agent is as described and relates to the chemical coating solution outlined in U.S. Pat. Nos. 5,756,126, 5,807,527, and 5,496,562. The disclosures of these patents are incorporated herein by reference. Adsorption of the chemical coating solution to the selected filter membrane results in the formation of the filter membrane of one embodiment of the invention.

More specifically, in one embodiment, the lysis agent may include a protein denaturing agent and a free radical trap. The denaturing reagent can be a surfactant that will denature proteins and the majority of any pathogenic organisms in the sample. Anionic detergents are examples of such denaturing reagents. The lysis agent can include a weak base, a chelating agent, and the anionic surfactant or detergent, and optionally uric acid and urate salt as discussed in detail in the above-cited U.S. Pat. No. 5,807,527. The disclosure of this patent is incorporated herein by reference. More preferably, the weak base can be a Tris, trishydroxymethyl methane, either as a free base or as the carbonate, and the chelating agent can be EDTA, and the anionic detergent can be sodium dodecyl sulfate. Other coatings having similar function can also be utilized in accordance with the present invention.

Alternatively, the substrate consists of a matrix and an anionic detergent affixed thereto. The anionic detergent can be selected from the group including sodium dodecyl sulfate (SDS). SDS can be obtained in various forms, such as the C₁₂ form and the lauryl sulfate. Other anionic detergents can be used, such as alky aryl sulphonates, sodium tetradecylsulphate long chain (fatty) alcohol sulphates, sodium 2-ethylhexysulphate olefine sulphates, sulphosuccinates or phosphate esters. The anionic detergent, such as the SDS, can be applied to the filter matrix at varying concentrations.

Generally, 5%-10% w/v SDS (for coating) can be used in accordance with the present invention. For example, a definite optimum SDS concentration has been achieved in the 5-7.5% w/v SDS concentration range for coating particular glass microfiber in order to enrich for and purify different plasmid populations directly from liquid cultures in a multi-well format, such formats being well known in the art.

In one embodiment, the lysis agent is disposed, sorbed, or otherwise associated with the dry solid medium of the present invention such that the medium and lysis agent function together to immobilize nucleic acid thereon through an action of cellular lysis of cells presented to the support. That is, the lysis agent can be adsorbed, absorbed, coated over, or otherwise disposed in functional relationship with the media. As stated above, the support or the present invention is preferably a porous filter media and can be in the form of a flat, dry media. The media can be combined with a binder, some examples of binders well-known in the art being polyvinylacrylamide, polyvinylacrylate, polyvinylalcohol, and gelatin.

The matrix of the present invention can be capable of releasing the generic material immobilized thereto by a heat elution. In a preferred embodiment, such a heat elution is accomplished by the exposure of the support having the genetic material stored thereon to heated water, the water being nuclease free.

The filter membrane of the invention is such that at any point during a storage regime, it allows for the rapid purification of immobilized nucleic acid. The immobilized nucleic acid is collected in the form of a soluble fraction following a simplified elution process, during which immobilized nucleic acid is released from the filter membrane of the invention. The filter membrane of the invention yields nucleic acid of sufficient quality that it does not impair downstream analyses such as polymerase chain reaction (PCR), ligase chain reaction (LCR), transcription mediated amplification (TMA), reverse transcriptase initiated PCR, DNA or RNA hybridization techniques, sequencing, and the like. Other post-purification techniques include cloning, hybridization protection assay, bacterial transformation, mammalian transfection, transcription-mediated amplification, and other such methods.

The nucleic acids retained by the filter may be washed with any suitable wash solution. Preferably, the nucleic acid retained by the filter is washed with a buffer having a pH in the range 5.8 to 10, more preferably in the range 7 to 8. In particular, washing with water or a low salt buffer such as TE⁻¹ (10 mM Tris HCl (pH8) with 100 μm EDTA) is preferred. The washing step may occur prior to or at the same time as elution. Washing increases the yield and purity of the nucleic acid product.

If desired, in some embodiments of the invention, it is possible to elute the nucleic acids from the filter. Elution may be performed at room temperature, but it is preferred to use heat treatment to increase the energy of the elution step. In a preferred embodiment of the invention, the elution step comprises heating the elution buffer to an elevated temperature prior to addition to the filter. In another preferred embodiment, the elution step comprises adding the elution buffer to the filter and then heating the filter with the elution buffer to an elevated temperature. In a more preferred embodiment, the elution step comprises heating the elution buffer to an elevated temperature prior to addition to the filter and then heating the filter with the elution buffer to an elevated temperature. Preferably, the elevated temperature is between 40° C. and 125° C. More preferably, the elevated temperature is between 80° C. and 95° C. Most preferably, the filter with the elution buffer is heated to an elevated temperature between 80° C. and 95° C. for 10 minutes.

Eluting the nucleic acid, in other words releasing the nucleic acid from the filter, may be affected in several ways. The efficiency of elution may be improved by putting energy into the system during an incubation step to release the nucleic acid prior to elution. This may be in the form of physical energy (for example by agitating) or heat energy. The incubation or release time may be shortened by increasing the quantity of energy put into the system.

Preferably, heat energy is put into the system by heating the nucleic acid to an elevated temperature for a predetermined time, while it is retained by the filter, prior to eluting, but not so hot or for such a time as to be damaged. [However, elution still may be effected when the nucleic acid has not been heated to an elevated temperature or even has been held at a lowered temperature (as low as 4° C.) prior to elution in step (e).] More preferably, the nucleic acid is heated to an elevated temperature in the range of 40° C. to 125° C., even more preferably in the range of from 80° C. to 95° C. Most preferably, the nucleic acid is heated to an elevated temperature of about 90° C., advantageously for about 10 minutes for a filter having a 6 mm diameter. Increasing the filter diameter increases the yield of DNA at any given heating temperature.

Once the nucleic acid has been heated to an elevated temperature while retained by the filter, it is not necessary to maintain the nucleic acid at the elevated temperature during elution. Elution itself may be at any temperature. For ease of processing, it is preferred that, where the nucleic acid is heated to an elevated temperature while retained by the filter, elution will be at a temperature lower than the elevated temperature. This is because when heating has been stopped, the temperature of the nucleic acid will fall over time and also will fall as a result of the application of any ambient temperature eluting solution to the filter. Preferred elution solutions include NaOH 1 mM to 1 M, Na acetate 1 mM to 1 M, 10 mM 2-[N-morpholino]-ethanesulfonic acid (MES) (pH 5.6), 10 mM 3-[cyclohexylamino]-1-propanesulfonic acid (CAPS) (pH 10.4), TE (10 mM Tris HCL (pH8)+1 mM EDTA), TE⁻¹ (10 mM Tris; 0.1 mM EDTA; pH 8), sodium dodecyl sulfate (SDS) (particularly 0.5% SDS), TWEEN™ 20 (particularly 1% TWEEN™ 20), LDS (particularly 1% lauryl dodecyl sulfate (LDS)) or TRITON™ (particularly 1% TRITON™), water and 10 mM Tris. Total yields of nucleic acid are higher when eluted in a high volume of elution solution.

The source of the nucleic acid can be a biological sample containing whole cells. The whole cells can be, but are not restricted to, blood, bacterial culture, bacterial colonies, saliva, urine, drinking water, plasma, stool samples, and sputum. The source can be a sample tube containing a liquid sample; an organ, such as a mouth, ear, or other part of a human or animal; a sample pool, such as a blood sample at a crime scene or the like; whole blood or leukocyte-reduced blood; or other various sources of cells known in the scientific, forensic, and other arts.

Cells from which nucleic acids are isolated may include both prokaryotic and eukaryotic cells, including oocysts, bacteria, and microbes. In addition, cells may be part of tissues or organisms, such as small monocellular or multicellular organisms. One example of a small organism is C. elegans. Cells, tissues, organs, or organisms may be treated, such as by homogenization, mincing, sonication, or isolation, prior to use according to the invention. Alternatively, viruses may be the source of the nucleic acids, or nucleic acids may be isolated from a non-cellular, non-viral sample.

In general, the present method may be applied advantageously to any whole cell suspension. Cells particularly amenable to the present method include bacterial cells yeast cells and mammalian cells, such as white blood cells, epithelial cells, buccal cells, tissue culture cells and colorectal cells.

Where the cells comprise white blood cells, it is preferred that the method further comprises applying whole blood to the solid phase, optionally lysing the red blood cells therefrom, optionally washing the solid phase to remove contaminants and obtaining the cell lysate from the blood cells. The whole blood can be fresh or frozen. Blood containing Na/EDTA, K/EDTA, and citrated blood all give similar yields. A 100 μl sample of whole blood gives a yield of approximately 2-5 μg of nucleic acids, a 500 μl sample gives a yield of approximately 15-40 μg of nucleic acids and a 10 ml sample gives a yield of approximately 200-400 μg of nucleic acids.

Preferably, the nucleic acid is either DNA or RNA, and most preferably it is DNA.

The present invention can find utility in many areas of genomics. For example, the present invention provides the capability to elute bound genetic material for the rapid purification of the genetic material to be utilized in any number of forensic applications, such as identification, paternity/maternity identification, and at the scene of a crime.

There are many liquids in several industries that should not have any biocontamination at point of sale. Also liquids are monitored for increase in biocontamination over time. Liquids may also include biological samples where the presence of microbes may illustrate disease or infection. A sample of a liquid would be added to a device of the invention, such as depicted in FIG. 1, to concentrate the cells or viruses in the liquid and subsequently isolate the nucleic acid. This type of system can be utilized in the food industry, with liquids including milk, wine, beer, and juices. It has valuable applications for concentrating wash water of agricultural products to test for bacterial contamination of these products. For example, fruits, vegetables, or meats may be rinsed with water, and the wash water may be tested for contamination.

In medicine, urine, blood, and stool extract can all be applied to the system with direct detection of the immobilized nucleic acid carried out with species-specific probes. In the environmental industry, analysis of drinking water, seawater, and river water can find utility within the proposed system.

### EXAMPLE 1

A standard GenSpin™ tube (Whatman, Inc.) is used. The tube has an FTA™ Elute filter and has a grid at the base of the spin basket below the bottom of the filter. The tube is inverted, and the grid, now on top, is removed to expose the FTA™ filter and also to form a cup to receive the nucleic acid containing sample.

A high-particulate sample containing nucleic acids is placed on the filter of the inverted spin basket and allowed to enter the filter material, thereby lysing the remaining cellular material, inactivating any pathogens present, and trapping any nucleic acids. The filter in the spin basket is then placed upright into the spin tube.
The filter is washed, preferably twice, with FTA™ buffer (0.5% weight-by-volume (w/v) sodium dodecyl sulfate (“SDS”) in H₂O) and centrifuged.
The filter is washed, preferably twice, with 10 mM Tris-HCl/1 mM EDTA/pH 8 (“TE”) and centrifuged.
50 μl DNase-free sterile water is added and the tube is heated, e.g., by being placed in boiling water for 10 minutes, followed by immediate centrifugation to elute and recover purified nucleic acids, such as DNA, for further use or archiving.
Preferably, the elution/recovery step is repeated at least once for improved yield.

### EXAMPLE 2

### Filtration without Gasket Assistance

Objective: To establish a basic unit by which the collection of cells from a large volume of solution, similar to what might be collected from washing a batch of fruit or vegetables, could be feasibly completed.

Method: A volume of “wash” was spiked with bacterial cells and processed over a dense pre-filter column to catch any large particulates. The resulting flow-through was then passed over a another filter unit containing only a track-etch membrane at the base. Filtration of this wash was followed by inversion of the membrane and subsequent collection of the cells (by vacuum; −20 in Hg), and deposited on the membrane in a small volume of media.

Results: Transformation data for the platings of the recovered sample show a low retrieval rate of the cells spiked into the original wash (see Table 1).

TABLE 1
Recovery without Gasket

| 500 cell spike | # colonies | recovery |
|---|---|---|
| trial 1 | 119 | 27% |
| trial 2 | 16 | 4% |
| trial 3 | 59 | 13% |
| 500 cell control | 448 | (=100%) |
| (straight plating) | | |

Conclusion: Although filtration with the single column unit is possible, only a small fraction of cells can be collected from the original spike of bacteria. A modification of the device would be necessary to improve recovery.

### EXAMPLE 3

### Filtration with Rubber Gasket Assistance

Objective: To implement the use of rubber gaskets in the construction of the basic unit and changes in the processing protocol to increase the recovery rate of bacterial cells spiked into a wash.

Method: Assuming that the low cell recoveries from Example 2 were due to flow of the “wash” solution around the track-etch membrane rather than through it, a rubber gasket placed on top of the track-etch membrane was implemented in the construction of the second filtration column. The gasket was rigid, ring-shaped, and a few millimeters thick. It was cut to fit snuggly inside the rim of the solid support. Several adaptations to the processing protocol were also made to help maximize cell recovery.

Results: Table 2 demonstrates that the use of the rubber gaskets is ineffective at improving cell recovery. Washing the collected cells from the track-etch membrane rather than inverted collection by vacuum is much easier. Arranging the pre-filter and second filter units in tandem also added to ease of operation.

TABLE 2
Recovery with Rubber Gasket

| | Plating of | Filter | Recovery |
|---|---|---|---|
| Protocol Variable | Wash | Plating | (Ave) |
| Standard | 12 | 15 | 2% |
| | 0 | 48 | 8% |
| Wash/No Flip | 7 | 4 | 1% |
| Filter | 24 | 72 | 8% |
| Plate Filter | x | 30 | 5% |
| Directly | x | 28 | 5% |
| Double Column | 11 | 27 | 3% |
| “piggyback” | 2 | 0 | 0.3% |
| 500 cell control | | | 609 (=100%) |
| (straight plating) | | | |

Conclusion: Using rubber gaskets to modify the assembly of the basic unit and a few protocol changes has not increased cell recovery significantly. Further modifications would be made to the system to increase recovery, but while the adjustments to the protocol do not improve cell recovery, they proved to be easier for the operator and will be adopted into future experimental design to streamline the process: namely manual wash vs. vacuum retrieval and the “piggyback” column arrangement.

### EXAMPLE 4

### Filtration with Polypropylene Gasket Assistance and Vacuum Change

Objective: To implement the use of polypropylene gaskets in the construction of the basic unit and to decrease the vacuum pressure for the processing protocol.

Method: After a quick test using a single rubber gasket to seal a track-etch membrane over a fritted glass funnel (creating a closed system) also lead to low cell retrieval, the hypothesis that high vacuum pressure may be damaging the bacterial cells was investigated. In addition, the material from which the gaskets were made was changed to polypropylene rather than rubber in hopes of creating a better seal if necessary.

Results: After tests using a single rubber gasket to seal a track-etch membrane over a fritted glass funnel (creating a closed system) also lead to low cell retrieval, the hypothesis that high vacuum pressure may be damaging the bacterial cells was investigated. In addition, the material from which the gaskets were made was changed to polypropylene rather than rubber. Results from processing a spiked wash using these new adaptations proved to be the ultimate for development of this device. The results show at least 50% cell recovery from the spiked wash. Two polypropylene gaskets sandwiching the track-etch membrane were used. These gaskets were very flexible, ring-shaped, and extremely thin (less than 1 mm thickness). They were cut to fit snuggly inside the rim of the solid support.

TABLE 3
Recovery with Polypropylene Gasket and Vacuum Change

| | | Recovery |
|---|---|---|
| | # colonies | (ave) |
| High vac (−25 in Hg) | 290 | 52% |
| High vac (−25 in Hg) | 559 | |
| Low Vac (−5 in Hg) | 600 | 82% |
| Low Vac (−5 in Hg) | 738 | |
| Negative Control | 0 | |
| 500 cell spike | 811 | (=100%) |
| control | | |

Conclusion: High vacuum pressure (−20 to 25 in Hg) was responsible for at least a portion of the poor cell retrieval results. The system must be operated under low vacuum (−5 in Hg is successful) to attain high cell recovery rates, and the use of polypropylene gaskets in the unit assembly offers even further increase in cell recovery rates (see Table 3).

### EXAMPLE 5

A device consisting of two filters in series is used. The first filter is a dense filter in a plastic funnel, which is used as a pre-filter. The funnel empties into an attached funnel containing a small-pore membrane, which acts as a size-exclusion barrier to trap the components of the mixture that contain nucleic acid. The trapped components are then removed from the surface and applied to FTA™, which is dried and washed for nucleic acid analysis.

A high particulate sample is pre-filtered through a dense matrix to remove large particulates. High particulate samples may be complex mixtures containing one or more of the following: cells, bacteria, oocysts, viruses, or other microbes. They may also contain sand, soil, or the like. The components of the mixture that pass through the pre-filter are trapped on the surface of the small-pore membrane. The samples are then collected for nucleic acid purification, either by washing the sample off the surface of the small-pore membrane in a small amount of isotonic buffer of neutral pH and then applying the washes to an FTA™ filter, or by swabbing the surface of the small-pore membrane with a small piece of FTA™ filter. The samples applied to the FTA™ filter are allowed to dry.

For nucleic acid purification, a small (2 mm) punch is taken of the region of the filter where there is applied samples that had been washed from the surface of the small-pore membrane or the entire small piece of FTA™ filter that was used to swab the surface of the small-pore membrane is used. Twice the filter is washed with FTA™ buffer (0.5% w/v SDS). Twice the filter is washed with TE.

The nucleic acid may be analyzed by PCR amplification or an alternative procedure. For example, PCR amplification may be of a DNA fragment of interest (genomic, plasmid, or otherwise, including viral DNA) or may be of a sequence from a housekeeping gene. Multiple round of amplification may be performed to increase sensitivity.

Here two primers were used to amplify a 1.7 kb enolase gene product:

Primer Sequences for Amplification of Enolase:

| | |
|---|---|
| Enolase primer #1 (forward) | |
| 5′ | |
| ATG TCC AAA ATC GTA AAA ATC ATC 3′ | (SEQ ID NO. 1) |
| Enolase primer #2 (reverse) | |
| 5′ TCA GAT AAT GTC AGT CTT ATG 3′ | (SEQ ID NO. 2) |

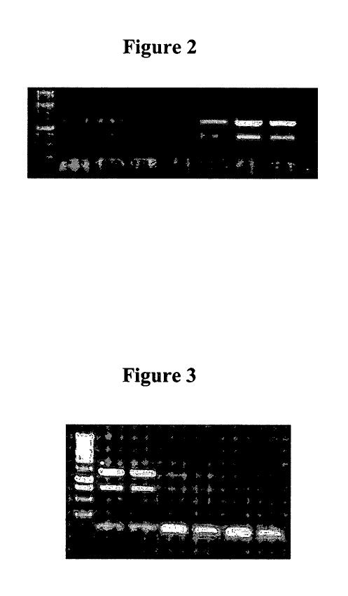

A mixture of these two primers with water in a total of 100 μl was added to 4 Amersham Ready-to-Go™ PCR beads and subjected to the following thermal cycling program: 94° C. for 3 min., then 94° C. for 30 secs., 55° C. for 30 secs., and 72° C. for 3 min. 30 secs. (for a total of 30 cycles), and a final 72° C. for 15 min. The results are pictured in FIG. 2 (M=molecular weight marker; lanes 1-2=positive enolase PCR controls; lanes 3-4=negative PCR controls; lanes 5-7=PCR of bacteria (large number of cells) collected on FTA™ filter). Bands are visible for the positive controls and for the PCR of bacterial DNA in lanes 5-7.

### EXAMPLE 6

### Purification of Nucleic Acid from Suspensions of Particulate Material Including Cells, Oocysts, and Bacteria from Washings of Foods

This example provides a method for the isolation of nucleic acid from cells, bacteria, oocysts and other microbes that are suspended in a large volume. The system utilizes a rapid pre-filtration and specific whole cell capture step coupled with FTA™ processing (Whatman, Inc.) to provide a fast and simple method to provide nucleic acid for analysis.

Description: This is for the isolation of nucleic acid from suspensions of materials washed from foods. Typically, these samples can be heavily particulated due to the presence of soil on the food and consequently in the washes. The device consists of two components, a filtration funnel assembly for the concentration of the sample and an FTA™ filter (or on a piece of FTA™, such as an FTA™ swab) for the isolation and purification of nucleic acid from the concentrated sample. The procedure is done in two stages:
Stage 1) The concentration of sample from food washings by filtration. This includes:
   (a) A prefiltration step to remove large particulates and
   (b) A filtration of the flow-through to capture and concentrate the microbes present in the suspension.
Stage 2) The application of the concentrated sample to FTA™ filters for the isolation of nucleic acid for the detection and analysis of the microbes present in the suspension.
   Stage 1: Concentrating Cells, Bacteria, Oocysts or other Microbes from Large Volumes of Liquid that Contain Particulates Such as Soil.

Brief overview: This filtration device is designed to provide a simple and rapid means of concentrating bacteria or other microbes in a sample from a volume of 50-500 ml down to 0.5 ml or less for the application to FTA™. The complete device consists of two sterile filter units that are connected in series. The first unit is a pre-filter funnel that catches large particulates but allows suspended cells, bacteria, oocysts and other microbes to pass through. The second unit is a 0.2 μm pore membrane filter funnel, which traps the bacteria on the surface where they can be (a) washed off with a small volume of an isotonic buffer for application to FTA™ filters, or (b) wiped with a small piece of FTA™. The FTA™ is then used for nucleic acid analysis.

Materials:

A particulate capturing pre-filter funnel containing a glass matrix filter (BS2000 Filter).
A bacterial filter funnel containing 0.2 μm polycarbonate track-etch filter membrane with polypropylene gaskets.
A silicone rubber gasket to make a seal between the device and the filtration flask.
An FTA™ filter, full-sized or cut into small (2-7 mm diameter) pieces for removal of the microbes trapped on the surface of the track-etch membrane.
   Additional Materials Required:
Vacuum pump (either a mechanical pump, a house vacuum line or water aspiration)
Side Arm vacuum flask (capacity≧500 ml.)
Isotonic Buffer (such as 1X phosphate-buffered saline (“PBS”; 10×=137 mM NaCl; 2.7 mM KCl; 5.4 mM Na₂HPO₄; 1.8 mM KH₂PO₄; pH 7.4)) or other medium for washing bacteria or other microbes off the surface of the filter membrane.
   Detailed Procedure:
   Assembly of the Device.

Each filter funnel in the device, the pre-filter and the bacterial filter, contains a filter and has an outlet end, such as a Luer outlet end. The outlet end of the pre-filter unit is inserted into the open end of the bacterial (size-exclusion) filter unit. During filtration the sample flows through the pre-filter into the bacterial filter unit. The two tubes fit together snugly.

The precut rubber gasket is laid on top of the opening of a side arm filter flask to provide an airtight seal during the vacuum filtration step.

The assembled device is placed onto the rubber gasket so that the bottom outlet empties into the vacuum flask. Use of a Luer outlet may improve the efficiency of the vacuum filtration.

An example of the device (10) is provided in FIGS. 1A and 1B. In FIGS. 1A and 1B, the arrows indicate the direction of the sample flow. Preferably, a vacuum is applied to the device to improve the rate of flow. The upper funnel (20) contains the dense pre-filter (22), which is supported by a support (24). The sample is added to the upper funnel (20), and the large particulates, such as those found in soil, are trapped in the pre-filter (22), while the target sample flows through the pre-filter (22) and through the outlet (26) into the lower funnel (30) containing the small-pore membrane (size-exclusion barrier) (40), which is supported by a support (42). Optionally, the small-pore membrane is sandwished between two ring-shaped gaskets (44 and 46). Use of the gaskets may improve results. The pre-filter (22) is washed with a small amount of isotonic buffer of neutral pH to minimize any retention of target sample. The small-pore membrane (40) acts as a size exclusion barrier, allowing the liquid to pass through the small-pore membrane (40) and the outlet (48), but trapping the particles, which include one or more of the following: cells, bacteria, viruses, oocysts, and other microbes, as well as other similar-sized particulates in suspension. The device may be disassembled and the sample collected from the surface of the small-pore membrane and applied to an FTA™ filter (or a piece of FTA™) as described below.

Filtration of the Sample.

The sample to be filtered is poured into the pre-filter funnel. Almost immediately liquid should begin to drip into the lower funnel unit.

Vacuum is applied to draw the sample through the device. In the experiments, the best results were obtained when using low vacuum pressure, 8″ to 10″ Hg (=200 to 250 mm Hg), however there was also some success (although with less reproducibility) when using higher vacuum pressure, 20″ Hg (=500 mm Hg). Note: If the vacuum does not have a gauge, the flow rate from the lower unit should be approximately 10 ml in 30 seconds.

If a very large volume of liquid is being filtered, it can be added in stages to the pre-filter funnel. Liquid is added to the upper funnel until all of the sample has been filtered through the device.

Any bacteria or microbes that may have been trapped in the pre-filter are removed by washing the inside walls of the pre-filter tube with additional amounts of buffer.

The pre-filter is completely dried by allowing air to be drawn through the filter apparatus for approximately 10 seconds after the liquid has finished dripping from the Luer end.

The vacuum is turned off, the upper (pre-filter) funnel unit is removed and the inside walls of the lower funnel are washed gently with 2-3 ml of sterile buffer. Vacuum is reapplied until after the liquid has completely drained. Air is drawn through the device for another 10 seconds to completely remove any excess liquid.

Stage 2: Collection of Sample from the Membrane for Application to FTA™:

Sample is collected using either of two methods:
(1) Trapped cells, bacteria, oocysts and other microbes are collected by rinsing the surface of the membrane filter with a small volume of buffer with a hand held pipettor or similar device. Two small washings have been used and been combined. The washings can then be applied to FTA™.
(2) Trapped bacteria or oocysts are collected by wiping the surface of the track-etch membrane with a small piece of FTA™ filter (a punch of 2-7 mm diameter).

The FTA™ that has had sample applied is then dried and processed in the normal manner for purification and analysis of nucleic acid. PCR or another type of analysis may then be performed.

Results:

Recoveries of 75-82% have been obtained when using 100 or 500 (E. coli) cells to spike a 200 ml sample of sterile 0.9% (w/v) saline (containing a small amount of autoclaved soil). For example, results of PCR reactions performed according to the method described in Example 5, using the nucleic acid from different numbers of cells on the FTA™ as a template, are shown in FIG. 3 (M=molecular weight marker; lanes 1-6=enolase PCR products (lanes 1-2=5×10⁶ cells; lanes 3-4=1×10⁴ cells; lanes 5-6=1×10² cells)). Primers used were those described in Example 5 (above).

When using a nested PCR protocol (which includes 2 rounds of PCR amplification), as few as 12 bacteria that have been spotted onto FTA™ have been detected, as described in Example 7 below.

Approximately 10% of the cells remain on the surface of the filter after the washings.

This device may be used to filter a variety of samples, including homogenized produce.

The advantages of this method and device are as follows:

(1) The process, from starting material to nucleic acid that is ready for analysis, is extremely rapid. The total time of the procedure, from application of raw sample to analysis of nucleic acid, can be measured in minutes.
(2) The methodology is simple, there are no specialized techniques to learn, nor is there a need for complicated lab equipment. The approach is very straightforward with few manipulations.
(3) Samples can be collected in the field. Once the samples are applied to FTA™, the nucleic acid is safe and can be analyzed immediately or it can be archived for analysis later.
(4) All of the necessary materials and equipment are easily available. There is no need of centrifugation.
(5) There are no dangerous chemicals or materials of any kind to deal with. Both the FTA™ and the filtration components of the device are safe and non-hazardous to the personnel collecting the samples or processing them.

### EXAMPLE 7

### PCR Detection of Bacterial Cells

Objective: To detect the cells collected from a spiked wash solution via PCR and determine the limits of sensitivity.

Method: A two-step PCR amplification of the enolase gene product was performed using nested primers.

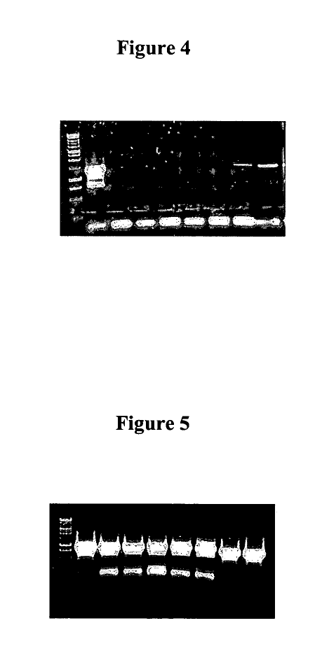

Results: FIG. 4 shows the first round results of PCR on the DNA in cells collected onto FTA membrane and amplified with primers for the enolase gene product. M indicates the molecular weight marker. Lanes 1-6 represent enolase PCR on cells collected on a FTA™ filter (1=concentrated culture; 2=1200 cells, 3=2300 cells; 4=1000 cells; 5=200 cells; 6=12 cells (counts are averages)). Lanes 7 and 8 are positive controls. PCR product from the concentrated sample, representing a very high number of cells, is the only one detecteable at this point. Primers used were those described in Example 5 (above).

However, when part of this amplification is used as template in a subsequent reaction, DNA from as few as 12 bacterial cells can be detected. FIG. 5 shows the second round results of PCR re-amplification of the first round PCR products with enolase gene product primers internal to those used in the first round of PCR. Lanes 1-8 correspond to lanes 1-8 of FIG. 4. M indicates the molecular weight marker. Lanes 1-6 represent enolase PCR on cells collected on a FTA™ filter (1=concentrated culture; 2=1200 cells, 3=2300 cells; 4=1000 cells; 5=200 cells; 6=12 cells (counts are averages)). Lanes 7 and 8 are positive controls.

The following nested primers were used:

Nested Primers for the Second Amplification of Enolase:

| | |
|---|---|
| (Forward) | |
| 5′ TCG ATA CGA ATC AGC TGG 3′ | (SEQ ID NO. 3) |
| (Reverse) | |
| 5′ TGA CAA GAT CAT GAT CGA CC 3′ | (SEQ ID NO. 4) |

Conclusion: Detection of a large number of bacteria collected onto to FTA is possible with one round of PCR. But sensitivity is dramatically improved with a second round of PCR, refining detection of thousands down to tens of cells.

### REFERENCES

Lampel, Keith A., et al. Improved Template Preparation for PCR-Based Assays for Detection of Food-Borne Bacterial Pathogens. Appl. Env. Microbiol. 66(10): 4539-4542 (2000).
Higgins, James A., et al. Detection of Francisella tularensis in Infected Mammals and Vectors Using a Probe-Based Polymerase Chain Reaction. Am. J. Trop. Med. Hyg. 62(2): 310-318 (2000).
Orlandi, Palmer A., and Lampel, Keith A. Extraction-Free, Filter-Based Template Preparation for the Rapid and Sensitive PCR Detection of Pathogenic Parasitic Protozoa. J. Clin. Microbiol. 38: 2271-2277 (2000).

1. A method of isolating nucleic acids from a sample containing cells or viruses, comprising:
a. providing a dry solid medium comprising a composition containing a lysis agent;
b. contacting the medium on one surface with the sample;
c. lysing the cells or viruses and allowing components of the sample, comprising the nucleic acids, to enter the medium;
d. washing the medium from the opposite surface with a wash buffer; and
e. eluting the nucleic acid from the medium.

2. The method of claim 1, wherein the lysis agent of step a comprises an anionic surfactant or an anionic detergent.

3. The method of claim 2, wherein the lysis agent further comprises:
i. a weak base;
ii. a chelating agent; and
iii. optionally uric acid or a urate salt.

4. The method of claim 1, wherein the dry solid medium comprises glass fiber, cellulose, or non-woven polyester.

5. The method of claim 1, wherein the dry solid medium is in the form of a swab or a filter.

6. The method of claim 1, wherein the eluting step d further comprises
i. heating an elution buffer to an elevated temperature in the range of 40° C. to 125° C.; and
ii. contacting the medium with the heated elution buffer.

7. The method of claim 6, wherein the elevated temperature is in the range of 80° C. to 95° C.

8. The method of claim 1, wherein the eluting step d further comprises:
i. contacting the medium with an elution buffer; and
ii. heating the medium and the elution buffer to an elevated temperature in the range of 40° C. to 125° C.

9. The method of claim 8, wherein the elevated temperature is in the range of 80° C. to 95° C.

10. The method of claim 8, wherein the heating step ii further comprises incubation for 10 minutes at the elevated temperature.

11. The method of claim 1, wherein the nucleic acids comprise DNA or RNA.

12. The method of claim 1, wherein the sample comprises a biological tissue or organ, a cell, a virus, a homogenate of a biological tissue or organ, blood, bile, pus, lymph, spinal fluid, feces, saliva, sputum, mucus, urine, discharge, tears, sweat, culture medium, water, wash water, or a beverage.

13. A method of isolating nucleic acids from a sample containing cells or viruses, comprising:
a. providing a dry solid medium;
b. lysing the cells or viruses with a lysis agent;
c. contacting the medium on one surface with the lysed sample to allow components of the sample, comprising the nucleic acids, to enter the medium;
d. washing the medium from the opposite surface with a wash buffer; and
e. eluting the nucleic acid from the medium.

14. The method of claim 13, wherein the lysis agent of step a comprises an anionic surfactant or an anionic detergent.

15. The method of claim 13, wherein the lysis agent further comprises:
i. a weak base;
ii. a chelating agent; and
iii. optionally uric acid or a urate salt.

16. The method of claim 13, wherein the medium comprises glass fiber, cellulose, or non-woven polyester.

17. The method of claim 13, wherein the dry solid medium is in the form of a swab or a filter.

18. The method of claim 13, wherein the eluting step e further comprises
i. heating an elution buffer to an elevated temperature in the range of 40° C. to 125° C.; and
ii. contacting the medium with the heated elution buffer.

19. The method of claim 18, wherein the elevated temperature is in the range of 80° C. to 95° C.

20. The method of claim 13, wherein the eluting step e further comprises:
i. contacting the medium with an elution buffer; and
ii. heating the medium and the elution buffer to an elevated temperature in the range of 40° C. to 125° C.

21. The method of claim 20, wherein the elevated temperature is in the range of 80° C. to 95° C.

22. The method of claim 20, wherein the heating step ii further comprises incubation for 10 minutes at the elevated temperature.

23. The method of claim 13, wherein the nucleic acids comprise DNA or RNA.

24. The method of claim 13, wherein the sample comprises a biological tissue or organ, a cell, a virus, a homogenate of a biological tissue or organ, blood, bile, pus, lymph, spinal fluid, feces, saliva, sputum, mucus, urine, discharge, tears, sweat, culture medium, water, wash water, or a beverage.

25. A method of isolating nucleic acids from a sample, comprising:
a. providing a dry solid medium comprising a composition consisting essentially of an anionic surfactant or an anionic detergent;
b. contacting the medium on one surface with the sample to allow components of the sample, comprising the nucleic acids, to enter the medium;
c. washing the medium from the opposite surface with a wash buffer; and
d. eluting the nucleic acid from the medium.

26. The method of claim 25, wherein the composition of step a further comprises:
i. a weak base;
ii. a chelating agent; and
iii. optionally uric acid or a urate salt.

27. The method of claim 25, wherein the dry solid medium comprises glass fiber, cellulose, or non-woven polyester.

28. The method of claim 25, wherein the dry solid medium is in the form of a swab or a filter.

29. The method of claim 25, wherein the eluting step d further comprises
i. heating an elution buffer to an elevated temperature in the range of 40° C. to 125° C.; and
ii. contacting the medium with the heated elution buffer.

30. The method of claim 29, wherein the elevated temperature is in the range of 80° C. to 95° C.

31. The method of claim 25, wherein the eluting step d further comprises:
i. contacting the medium with an elution buffer; and
ii. heating the medium and the elution buffer to an elevated temperature in the range of 40° C. to 125° C.

32. The method of claim 31, wherein the elevated temperature is in the range of 80° C. to 95° C.

33. The method of claim 31, wherein the heating step ii further comprises incubation for 10 minutes at the elevated temperature.

34. The method of claim 25, wherein the nucleic acids comprise DNA or RNA.

35. The method of claim 25, wherein the sample comprises a biological tissue or organ, a cell, a virus, a homogenate of a biological tissue or organ, blood, bile, pus, lymph, spinal fluid, feces, saliva, sputum, mucus, urine, discharge, tears, sweat, culture medium, water, wash water, or a beverage.

36. A method of isolating nucleic acid from a sample containing cells or viruses containing nucleic acid, comprising:
a. providing a pre-filter comprising a dense medium capable of retaining contaminants larger than the cells or viruses containing nucleic acid;
b. providing a size-exclusion barrier capable of retaining the cells or viruses containing nucleic acid;
c. contacting the pre-filter with the sample;
d. drawing the sample through the pre-filter so that the nucleic acid-containing cells or viruses are drawn through the filter;
e. contacting the size-exclusion barrier with the sample containing the nucleic acid-containing cells or viruses;
f. trapping the nucleic acid-containing cells or viruses on the size-exclusion barrier while drawing liquid components through the size-exclusion barrier; and
g. removing the trapped nucleic acid-containing cells or viruses from the filter.

37. The method of claim 36, further comprising:
h. providing a dry solid medium comprising a composition containing a lysis agent;
i. contacting the nucleic acid-containing cells or viruses with the medium;
j. lysing the nucleic acid-containing cells or viruses and allowing components of the sample, comprising the nucleic acids, to enter the medium;
k. washing the medium; and
l. eluting the nucleic acid from the medium.

38. The method of claim 37, wherein the lysis agent of step h comprises an anionic surfactant or an anionic detergent.

39. The method of claim 38, wherein the lysis agent further comprises:
i. a weak base;
ii. a chelating agent; and
iii. optionally uric acid or a urate salt.

40. The method of claim 37, wherein the dry solid medium comprises glass fiber, cellulose, or non-woven polyester.

41. The method of claim 37, wherein the dry solid medium is in the form of a swab or a filter.

42. The method of claim 37, wherein the eluting step 1 further comprises
i. heating an elution buffer to an elevated temperature in the range of 40° C. to 125° C.; and
ii. contacting the medium with the heated elution buffer.

43. The method of claim 42, wherein the elevated temperature is in the range of 80° C. to 95° C.

44. The method of claim 37, wherein the eluting step 1 further comprises:
i. contacting the medium with an elution buffer; and
ii. heating the medium and the elution buffer to an elevated temperature in the range of 40° C. to 125° C.

45. The method of claim 44, wherein the elevated temperature is in the range of 80° C. to 95° C.

46. The method of claim 44, wherein the heating step ii further comprises incubation for 10 minutes at the elevated temperature.

47. The method of claim 36, wherein the nucleic acid comprises DNA or RNA.

48. The method of claim 36, wherein the sample comprises a biological tissue or organ, a cell, a virus, a homogenate of a biological tissue or organ, blood, bile, pus, lymph, spinal fluid, feces, saliva, sputum, mucus, urine, discharge, tears, sweat, culture medium, water, wash water, or a beverage.

49. The method of claim 36, wherein the pre-filter of step a further comprises glass microfiber, cellulose acetate, polypropylene, melt-blown polypropylene, scintered glass, or polyethylene.

50. The method of claim 36, wherein the size-exclusion barrier comprises a polycarbonate track-etch membrane.

51-62. (canceled)
